(19) United States
(12) Patent Application Publication
(10) Pub. No.: US 2018/0011071 A1
(43) Pub. Date: Jan. 11, 2018

(54) METHOD FOR DETERMINING THE LIKELIHOOD RATIO FOR MEMBERSHIP IN TWO CLASSES BASED ON TARGETED COMPONENTS IN A SUBSTANCE

(71) Applicant: University of Central Florida Research Foundation, Inc., Orlando, FL (US)

(72) Inventors: Michael E. Sigman, Oviedo, FL (US); Mary R. Williams, Mount Dora, FL (US)

(21) Appl. No.: 15/643,316

(22) Filed: Jul. 6, 2017

Related U.S. Application Data
(60) Provisional application No. 62/359,093, filed on Jul. 6, 2016.

(51) Int. Cl. G01N 33/22; G06F 17/18

(52) U.S. Cl. CPC G01N 33/22; G06F 17/18

(57) ABSTRACT

Methods for determining the probability of finding a particular component of a mixture in a fire debris sample, conditioned on membership in each of the at least two distinct classes.

### INCORPORATION BY REFERENCE

The present application claims the benefit of and priority to U.S. Provisional Application Ser. No. 62/359,093, titled METHOD FOR DETERMINING THE LIKELIHOOD RATIO FOR MEMBERSHIP IN TWO CLASSES BASED ON TARGETED COMPONENTS IN A SUBSTANCE, filed Jul. 6, 2016, the entire disclosure of which is incorporated by reference into the present application.

### STATEMENT REGARDING FEDERALLY FUNDED RESEARCH OR DEVELOPMENT

This invention was made with Government support under National Institute of Justice (NIJ) award number NIJ-2015-3985(SL001149). The Government has rights in the claimed inventions.

### TECHNICAL FIELD

The presently disclosed and claimed inventive concept(s) relate to and method(s) for providing the probability of observing particular components of a mixture in an unknown sample. More specifically, the presently disclosed and claimed inventive concept(s) relate to the probability of observing particular components in an unknown sample conditioned on membership in two classes of substances.

### BACKGROUND

It is often necessary to analyze mixtures to determine what components they contain. This is true, for example, in the field of forensic science. Specifically, a forensic scientist may be called upon to analyze and successfully identify a sample from the scene of a fire or an explosion that has occurred. In the case of a fire, the scientist may need to determine (1) if an ignitable liquid is present; and (2) the type of ignitable liquid or substance present in a fire debris sample. In the case of an explosion, the scientist may need to identify or explain explosive materials that were used to cause the explosion. It is also desirable to assess the evidential value of the data.

There are various methods for identifying a particular chemical compound in a mixture, often by separation of the chemicals prior to identification. In other cases, however, it is necessary to identify the class to which a particular combination of chemicals pertains. For example, it may be desired to determine what class of ignitable liquid (e.g., gasoline, normal alkane, etc.) is present in a fire debris sample. In this example, gasoline is comprised of a combination of individual chemicals, and that combination of chemicals constitutes a component of the mixture. The mixture contains the component and additional chemicals that may comprise other components.

Existing identification methods in fire debris data analysis are not designed to make such component classifications in complex mixtures. Current practice to determine the presence of an ignitable liquid consists of visual pattern recognition applied to a sample's total ion chromatogram and extracted ion profiles. However, pattern recognition can become challenging because pyrolysis/combustion products from substrates are also extracted from the post-burn sample and detected by chromatography and some pyrolysis/combustion products may be identical to select ignitable liquid components. As such, pyrolysis of substrates can result in products that may be confused with ignitable liquid residue(s). Additionally, prior studies have shown that products from pyrolysis of substrate materials may also be mistaken for synthetic blends or specialty solvents. Despite the shortcomings of the current methodology, a large-scale cross tabulation between pyrolysis/combustion products and ignitable liquid components does not exist. Better performance is desired if these methods are to be implemented in casework and construed as having evidentiary value.

Accordingly, there is a need for an improved reference and/or substrate database to assist fire debris analysts in casework, to use the data in the development of an extensive cross tabulation of pyrolysis/combustion products with ignitable liquid components, and to also use the data to inform a more comprehensive understanding and modeling of fire debris samples. Better chemometric models will result in lower error rates (such as false positives and/or false negatives) and improved likelihood estimates for presence or absence of ignitable liquid residue in fire debris samples. More specifically, a naïve Bayesian likelihood ratio approach could provide the analyst with a more informative result by not only providing a classification of the sample, but also providing a measure of the strength of the evidence.

It can therefore be appreciated that it would be desirable to have an effective system and method for improved fire debris models to incorporate into chemometric methods that allow fire debris analysts to objectively assess the evidential value of a casework fire debris sample to determine the probability of finding a particular component of a mixture in a pyrolysis debris sample. It is to such methods that the presently disclosed and claimed inventive concept(s) is directed.

### DESCRIPTION OF THE SEVERAL VIEWS OF THE DRAWINGS

FIG. 1 is a block diagram of an embodiment of a system for providing the ratio of the probability of observing a plurality of components, conditioned on membership in each of two classes.

FIG. 2 is a block diagram of an embodiment of a computer shown in FIG. 1.

FIG. 3 is a flow diagram of a first embodiment of a method for providing the ratio of the probability of observing a plurality of components, conditioned on membership in each of two classes.

FIG. 4 is a three-dimensional graph that comprises various information regarding an analyzed sample, including the sample's total ion chromatogram, mass spectrum, and summed ion spectrum.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, devices, kits, and/or methods disclosed and claimed herein can be made and executed without undue experimentation in light of the present disclosure. While the compositions and methods of this presently disclosed and claimed inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The singular forms “a,” “an,” and “the” include plural referents unless the context clearly indicates otherwise. Thus, for example, reference to “a compound” may refer to 1 or more, 2 or more, 3 or more, 4 or more or greater numbers of compounds. The term “plurality” refers to “two or more.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by ±20% or ±10%, or ±5%, or ±1%, or ±0.1% from the specified value, as such variations are appropriate to perform the disclosed methods and as understood by persons having ordinary skill in the art. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “circuitry” as used herein includes, but is not limited to, analog and/or digital components (e.g., computers), in, or one or more suitably programmed processors (e.g., microprocessors) and associated hardware (e.g., input/output devices, or “I/O” devices) and software or hardwired logic. The term “digital components” may include hardware, such as but not limited to, processors (e.g., microprocessor), an application specific integrated circuit (ASIC), field programmable gate array (FPGA), a combination of hardware and software, and/or the like. The term “software” as used herein may include one or more computer readable medium (i.e., computer readable instructions) that when executed by one or more digital components cause the component to perform a specified function. It should be understood that the algorithms described herein may be stored on one or more non-transient memory. Non-limiting exemplary non-transient memory may include random access memory, read only memory, flash memory, and/or the like. Such non-transient memory may be electrically-based, optically-based, and/or the like. The term “processor” as used herein means a single processor or multiple processors working independently or together to collectively perform a task.

As used herein, the term “database” means a collection and/or library of data and/or reference records. Non-limiting records may include identifying information (e.g., spectral data) of samples.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

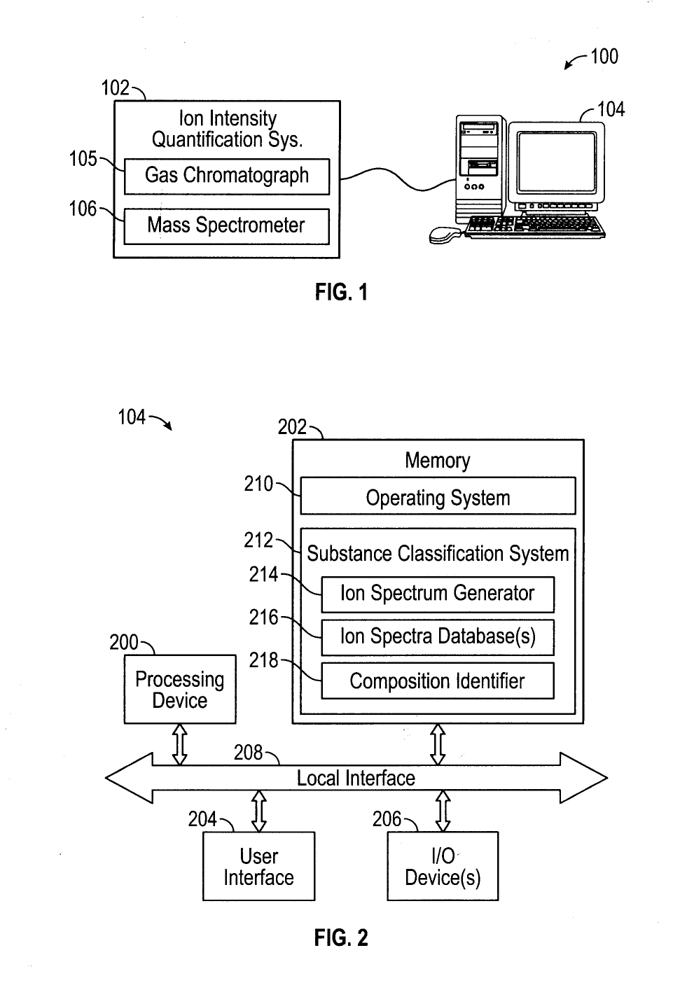

Referring now to the figures, and in particular to FIG. 1, shown therein is an example system 100 with which samples can be analyzed to provide the ratio of probability of observing a plurality of components, conditioned on membership in each of at least two distinct classes contained in the samples. As indicated in FIG. 1, the system 100 generally comprises an ion intensity quantification system 102 and a computer 104 that are coupled such that data can be sent from the data collection system to the computer. By way of example, the system 100 comprises part of a network, such as a local area network (LAN) or wide area network (WAN).

The ion intensity quantification system 102 is configured to quantify the intensity of ions resulting from compounds, such as those contained in test samples. In one non-limiting embodiment, the ion intensity quantification system 102 comprises a gas chromatograph 105 and a mass spectrometer 106 that together partially or fully separate the components of a given mixture down into various ions. Notably, the gas chromatograph and the mass spectrometer can be combined into a single apparatus (i.e., a GC/MS). In alternative embodiments of the presently disclosed and/or claimed inventive concept(s), the intensity quantification system 102 may include a laser spectrometer or infrared spectrometer.

As described below, the computer 104, and more particularly software provided on the computer, is configured to receive the intensity information from the ion intensity quantification system 102 and identify the chemical composition of the sample and the probability of observing the chemical composition conditioned on classes of substances that may be contained in the sample.

FIG. 2 is a block diagram illustrating a non-limiting embodiment of the architecture for the computer 104 of FIG. 1. In such non-limiting embodiment, the computer 104 comprises a processing device 200, memory 202, a user interface 204, and at least one I/O device 206, each of which is operationally connected to a local interface 208.

The processing device 200 can include a central processing unit (CPU) or a semiconductor-based microprocessor in the form of a microchip. The memory 202 includes any one of a combination of volatile memory elements (e.g., RAM) and nonvolatile memory elements (e.g., hard disk, ROM, etc.).

The user interface 204 comprises the components with which a user interacts with the computer 104 and therefore may comprise, for example, a keyboard, mouse, and/or a display. The one or more I/O devices 206 are adapted to facilitate communications with other devices or systems and may include one or more communication components such as a modulator/demodulator (e.g., modem), wireless (e.g., radio frequency (RF)) transceiver, network card, etc.

The memory 202 (i.e., a computer-readable medium) comprises various software programs including an operating system 210 and a substance classification system 212. The operating system 210 controls the execution of other programs and provides scheduling, input-output control, file and data management, memory management, and communication control and related services. As is indicated in FIG. 2, the substance classification system 212 comprises various modules, including an ion spectrum generator 214, one or more ion spectra databases 216, and a chemical composition identifier 218. Although each of those components are illustrated as being contained within in a single system 212 and stored on a single computer, it is noted that the components can be separated and/or distributed over two or more computers.

The ion spectrum generator 214 is configured to identify the chemicals present in the sample based on the ion intensities identified by the ion intensity quantification system 102 for all fractions of separated components of test samples.

The one or more ion spectra databases 216 comprise ion spectra for various chemical components of substances, such as ignitable liquids and explosive materials. Each substance may be associated with a given subclass of substances. For example but not by way of limitation, if the substances are part of the ignitable liquids class, each may be associated with a particular subclass, such as, by way of example and not by way of limitation, with an ASTM E1618 subclass. The ASTM subclasses includes aromatic (AR), gasoline (GAS), isoparaffinic (ISO), miscellaneous (MISC), normal alkane (NA), naphthenic paraffinic (NP), oxygenate (OXY), and petroleum distillate (PD). The stored spectra, and the chemicals which they represent, can be characterized according to the frequency with which they are observed in a class to which they pertain. In one non-limiting embodiment, the frequencies of chemical components identified by databases 216 can be stored in a separate database 216b on a separate computer that can be accessed using a network, such as the Internet. For example, the databases 216b can comprise central databases hosted by an official governing body (e.g., U.S. government) from which frequencies can be downloaded by analysts for the purpose of comparison with the chemical composition of collected samples.

The class identifier 218 is configured to compare the frequencies contained in the databases 216b with data associated with the chemical composition of unknown samples to provide the ratio of probability of observing the plurality of components, conditioned on membership in each of the distinct classes (i.e., the likelihood ratio).

Various programs (i.e., logic) have been described herein. Those programs can be stored on any computer-readable medium for use by or in connection with any computer-related system or method. With respect to the presently disclosed and/or claimed inventive concept(s), a computer-readable medium is an electronic, magnetic, optical, or other physical device or means that contains or stores a computer program for use by or in connection with a computer-related system or method. Those programs can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In view of the consistency of the mass spectra that are generated for given components, particularly when performing electron ionization at, for example but not by way of limitation, 70 electron-volts (eV), and therefore the consistency of chemical composition identification, unique combinations of chemical components, for example contained in substances from a collected sample, can be combined with the frequencies in databases 216b to provide an estimate of the probability of observing the plurality of components conditioned on class membership.

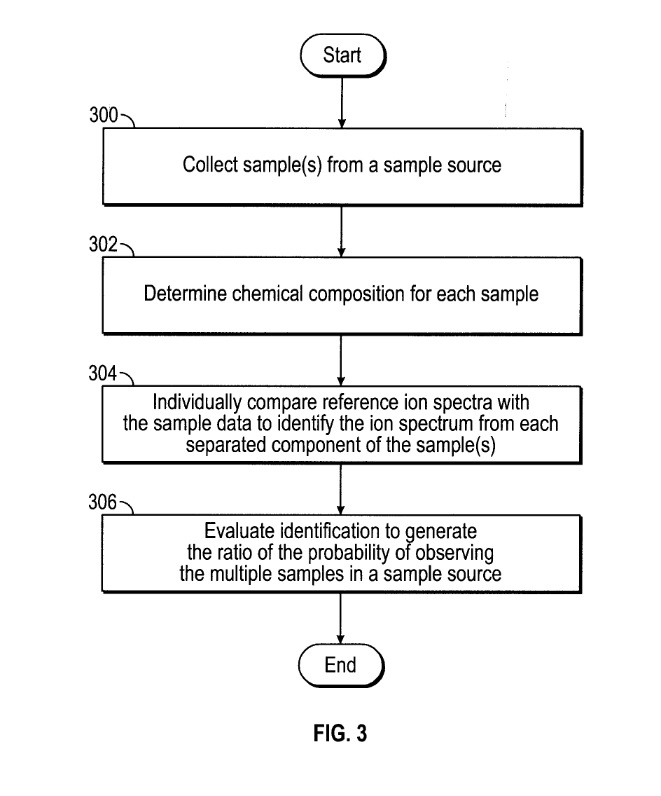

FIG. 3 provides a non-limiting embodiment of a method for providing the probability of observing a plurality of components, conditioned on membership in each of at least two distinct classes contained in the samples of a material (mixture) using chemical composition and the statistical relationship between the plurality of components of the sample and the frequency of components indicative of the at least two classes. As shown in block 300, one or more samples that are to be evaluated are collected from an unknown sample source. For example, when the evaluation is to be performed in relation to the scene of a fire, the samples can be debris samples collected from various locations at the fire scene. By way of example, the samples may comprise 5, 6, 7, 8, 9, 10, 11, 12, 13, 14, or 15 collected samples. Notably, each sample may have different concentrations of a substance (e.g., ignitable liquid) that was used to start the fire as well as background substrates (e.g., furniture, carpet, etc.) that were burned in the fire.

Next, as shown in block 302, the chemical composition can be determined for each sample. The various components of the sample can be separated using a gas chromatograph. During the separation, the various compounds contained within the sample elute at different times, resulting in a total ion chromatogram that plots the total detector response from ions detected as a function of time.

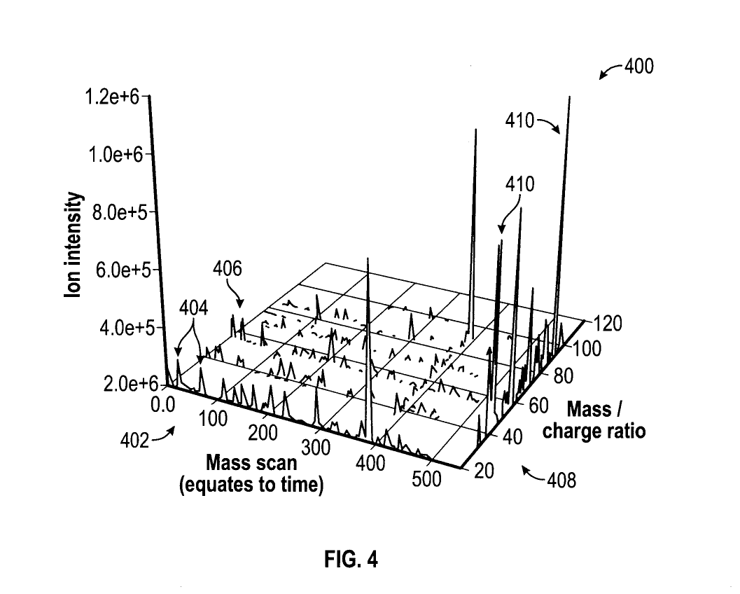

The three-dimensional graph 400 of FIG. 4 illustrates an example of a total ion chromatogram 402. As indicated in that figure, the total ion chromatogram 402 comprises multiple peaks 404, each pertaining to a different component (and its ions) that has been separated from the sample at a particular point in time.

The ion intensities from each of the components of the samples can be determined relative to their mass-to-charge ratios. In that process, the ions of each peak 404 of the total ion chromatogram 402 are analyzed to obtain an indication or representation of the number of ions for each of multiple mass-to-charge ratios. The ion intensities are identified as a function of mass-to-charge ratios in the graph 400 of FIG. 4 as a data set 406 (i.e., the peaks in the center of the x-y plane of the graph). In certain non-limiting embodiments, the ion intensities are determined using a mass spectrometer. In such a case, the various components can be received (e.g., from the gas chromatograph) by an ion source of the mass spectrometer that strips electrons from the component molecules to form positive ions, which then degrade into molecular fragments. The fragments that have a positive charge are then accelerated out from the ion source through a mass analyzer of the mass spectrometer, and into a detector that identifies ion intensities as a function of their mass-to-charge ratios.

The ion spectra from each separated component of the mixture can be identified based on comparison with ion spectra compiled in database 216.

Automated identification can be performed and all identifications can be made based on retention time match within a specified time window and a corresponding mass spectral match. One potential challenge to the automated process is that the mass spectral match must be made in the presence of partially co-eluting components. Several methods are available to accomplish this task, including but not limited to, those embodied in the Automated Mass Spectral Deconfolution and Identification System (“AMDIS”) software provided by the National Institute of Standards and Technology.

After the comparisons have been performed, the correlations of the substances in each substance class (e.g., ignitable liquid class) can be evaluated to determine the probability of observing a plurality of components, conditioned on membership in each of at least two distinct classes contained in the samples. As is described in greater detail below, this classification can be performed using Bayesian decision theory, for example, but not by way of limitation, a naïve Bayes model.

All of the targeted components are analyzed for mass spectral matches for each substance belonging to each class of substance. The number of occurrences of each targeted component in a class and the number of failures to observe each targeted component in a class are calculated.

After the comparisons have been made between the sample data and the reference substances, a certainty threshold must be met to determine whether the identified chemical is actually present. Logistic regression can be used to predict the probability that a targeted component is actually present in a substance. A set of comparison metrics for targeted components that are known to be members of substances, referred to as state 1, and targeted components that are not members of substances, referred to as state 0, are modeled by logistic regression. A cutoff value is set such that for any targeted component, if the probability calculated by logistic regression exceeds the cutoff, the targeted component is deemed to be present in the substance.

A database of targeted substances from each class is analyzed to determine the frequency of occurrence of each targeted component in each class. The frequency of occurrence, i.e., the number of occurrences divided by the number of substances in the class, is a maximum likelihood estimate of the probability of observing the targeted component given the class of substance. This probability, written as P(t|Sc), is the probability of observing target compound, t, given a class of substance, Sc.

Some targeted components may not be observed in a class due to the limited size of the database and the imperfect nature of the sample comprising the database. Rather than simply assigning a frequency of occurrence of zero to these targeted components, it is useful to predict their frequency of occurrence using Good-Turing numbers. The Good-Turing number, PO, gives the composite probability of observing all of the target components that were not observed for a class. The composite PO can be distributed over all of the targeted components in any fashion that makes sense for the data under analysis. The composite PO may be equally distributed over the targeted components that were not observed if no other \ reason exists to alter the distribution of PO.

After the comparisons have been made between the sample data and the reference substances and the frequency of occurrence of each targeted component in each class have been determined, a likelihood ratio may be established to provide a classification of the sample and assess the strength of the evidence. The likelihood ratio deals with the likelihood of observing the evidence under two exclusive hypotheses.

For classes of substances where the targeted components are shown to be independent, the probability of observing a set of targeted components is equal to the product of the probabilities of observing each targeted component(s). A set of targeted components {tl, t2 . . . } will be signified as E, which evidences a substance belongs to a specific class. The probability of observing the evidence given a substance class, Sc, can be expressed mathematically as in Equation 1, where Pli is the product over the index i, and where ti represents a set of i targeted components that are observed for a sample of a substance.

P(E|Sc)=Pli[P(ti|Sc)] (1)

The likelihood ratio for a substance belonging to class 1, S1, as opposed to class 2, S2, is given by Equation 2, where LR is the likelihood ratio.

LR=P(E|S1)/P(E|S2) (2)

After obtaining the likelihood ratio, a verbal scale can be readily applied to aid in expressing the evidential value of the data. This approach can (1) decrease and/or remove the human error involved with making categorical declarations, (e.g., regarding the presence or absence of an ignitable liquid); and (2) provide a natural evaluation of the strength of the evidence.

The systems and methods disclosed above provide a decision tool that can be automated, if desired. The systems and methods can be applied to the interpretation of complex samples in a laboratory, interpretation of sensor data in laboratory or field-deployed instruments, and process and manufacturing control. Areas of application for the systems and methods include forensic science (complex mixture classification), medicine (disease or pathogen classification), security applications (threat classification), and the like.

### NON-LIMITING EXAMPLES OF THE INVENTIVE CONCEPT(S)

A computer-implemented method for generating a likelihood ratio report, the method comprising the steps of: receiving data indicative of a plurality of components of a sample; comparing the plurality of components of the sample with stored data in a database, the stored data in the database comprising information related to known components indicative of at least two distinct classes; establishing a statistical relationship between the plurality of components of the sample and the known components indicative of the at least two classes to thereby provide the ratio of probability of observing the plurality of components, conditioned on membership in each of the at least two distinct classes; and generating, based at least in part on the statistical relationship between the plurality of components of the sample and the known components indicative of the at least two classes, a likelihood ratio report, wherein the likelihood ratio report includes a ratio of probability of observing the plurality of components, conditioned on membership in each of the at least two distinct classes.

The method, wherein the plurality of components are subclasses of substances of the sample.

The method, wherein the sample is debris from a scene of at least one of a fire and an explosion.

The method, wherein the stored data is spectral data selected from the group consisting of ion intensity data, chromatography fraction data, and total ion spectral data.

The method, wherein the data is ion intensity information obtained from the component of the mixture in the sample.

The method, wherein likelihood ratio report is digital data that can be displayed, transmitted, or printed.

The method, wherein comparing, by the instruction execution system, the the plurality of components of the sample with stored data in a database generates comparisons, and wherein sets of correlations are obtained from the comparisons, one set for each substance class, and wherein determining which substance class most closely correlates to the sample data comprises determining which set of correlations correlates most closely to the sample data using Bayesian decision theory.

Thus, in accordance with the presently disclosed and/or claimed inventive concept(s), there have been provided methods of an effective system and method for improved fire debris models to incorporate into chemometric methods that allow fire debris analysts to objectively assess the evidential value of a casework fire debris sample to determine the probability of finding a particular component of a mixture in a pyrolysis debris sample, conditioned on membership in each of the at least two distinct classes (i.e., the “likelihood ratio” and reports related thereto), which fully satisfy the objectives and advantages set forth hereinabove. Although the inventive concept(s) has been described in conjunction with the specific language set forth hereinabove, it is evident that many alternatives, modifications, and variations will be apparent to those skilled in the art. Accordingly, it is intended to embrace all such alternatives, modifications, and variations that fall within the spirit and broad scope of the presently disclosed and/or claimed inventive concept(s).

What is claimed is:

1. A computer-implemented method for generating a likelihood ratio report, the method comprising the steps of:
receiving data indicative of a plurality of components of a sample;
comparing the plurality of components of the sample with stored data in a database, the stored data in the database comprising information related to known components indicative of at least two distinct classes;
establishing a statistical relationship between the plurality of components of the sample and the known components indicative of the at least two classes to thereby provide the ratio of probability of observing the plurality of components, conditioned on membership in each of the at least two distinct classes; and
generating, based at least in part on the statistical relationship between the plurality of components of the sample and the known components indicative of the at least two classes, a likelihood ratio report, wherein the likelihood ratio report includes a ratio of probability of observing the plurality of components, conditioned on membership in each of the at least two distinct classes.

2. The method of claim 1, wherein the plurality of components are subclasses of substances of the sample.

3. The method of claim 1, wherein the sample is debris from a scene of at least one of a fire and an explosion.

4. The method of claim 1, wherein the stored data is spectral data selected from the group consisting of ion intensity data, chromatography fraction data, and total ion spectral data.

5. The method of claim 1, wherein the data is ion intensity data obtained from the plurality of components of the sample.

6. The method of claim 1, wherein likelihood ratio report is digital data that can be displayed, transmitted, or printed.

7. The method of claim 1, wherein comparing, by the instruction execution system, the the plurality of components of the sample with stored data in a database generates comparisons, and wherein sets of correlations are obtained from the comparisons, one set for each substance class, and wherein determining which substance class most closely correlates to the sample data comprises determining which set of correlations correlates most closely to the sample data using Bayesian decision theory.
